(19) United States
(12) Patent Application Publication
(10) Pub. No.: US 2013/0273571 A1
(43) Pub. Date: Oct. 17, 2013

(54) METHOD OF FUNCTIONALIZING HUMAN RED BLOOD CELLS WITH ANTIBODY

(71) Applicants: Christopher Knutson, Woodridge, IL (US); Derek David Doorneweerd, Lockport, IL (US)

(72) Inventors: Christopher Knutson, Woodridge, IL (US); Derek David Doorneweerd, Lockport, IL (US)

(73) Assignee: ARRYX, INC., Chicago, IL (US)

(21) Appl. No.: 13/696,171

(22) PCT Filed: May 5, 2011
(86) PCT No.: PCT/US11/00788

§ 371 Date: Jun. 20, 2013

Related U.S. Application Data
(60) Provisional application No. 61/282,997, filed on May 5, 2010.

(51) Int. Cl. G01N 33/555

(52) U.S. Cl. CPC G01N 33/555
USPC 435/7.25

(57) ABSTRACT

The present invention relates to producing more densely functionalized indicator cells which along, with other components, can be used to detect the presence or absence of antibodies or antigens, by using the A, B, AB and MNS antigens which have a greater degree of expression than the conventionally used D antigen to label the indicator cells with IgG. The present antigen systems have levels of expression that approach one million antigens per cell, which is in great contrast to the conventional D antigen sites of about 10,000-30,000 antigens per cell. This marked increase in the antigen systems level of expression could produce a boost in the kinetics and the magnitude of the binding of the indicator cell to the solid phase, which improves assay performance.

### CROSS-REFERENCE TO RELATED APPLICATIONS

The present invention claims priority from U.S. Provisional Application No. 61/282,997, filed May 5, 2010, which is herein incorporated by reference in its entirety.

### BACKGROUND OF THE INVENTION

1. Field of the Invention

The present invention relates to methods and apparatuses for functionalizing human red blood cells (RBCs) with antibody, and is relevant in the context of a solid-phase antibody screening assay. The present invention produces more densely functionalized indicator cells which, along with other components, can be used to detect the presence or absence of antibodies or antigens by targeting the A, B, AB and MNS antigens which have a greater degree of expression than the conventionally used D antigen. Further, the present invention may be extended to any immunoassay where one wishes to detect specifically bound immunoglobulin.

2. Description of the Related Art

Conventional solid-phase antibody screening assays detect IgG antibodies by relying upon anti-IgG coated red blood cells adhering or agglutinating to immunoglobulin that is specifically bound to a solid phase, for example. See for example, U.S. Pat. No. 4,816,413 to Sinor et al.

Conventional solid-phase antibody screening assays typically rely on indicators created by first sensitizing human red blood cells 10 (blood type of O+) with monoclonal or polyclonal anti-D antibodies 11. The D antigen 12 is used as sensitization of the red blood cells 10 occurs if D antigen 12 is present on the red blood cell 10. The IgG sensitized cells are then exposed to anti-IgG which renders the cells 10 anti-IgG coated.

The D antigen 12 is typically used because of the availability of high affinity anti-D antibody 11. However, the D antigen 12 is not highly expressed (10,000-30,000 antigens 12 per cell 10). In a solid-phase geometry, the area of interaction between a cell 10 and a planar surface can limit the interaction area such that only 10-100 anti-IgG molecules are interacting with the substrate. This limitation may strongly affect the kinetics and magnitude of binding of these indicator cells to the solid-phase.

Thus, a method and apparatus which produces more densely functionalized indicator cells which can be used to detect the presence or absence of antibodies or antigens, is needed.

### SUMMARY OF THE INVENTION

The present invention relates to producing more densely functionalized indicator cells which can be used to detect the presence or absence of antibodies or antigens, by using the A, B, AB and MNS antigens which have a greater degree of expression than the D antigen. These antigen systems have levels of expression that approach 1 million antigens per cell, which is in great contrast to the conventional D antigen sites of about 10,000-30,000 antigens per cell. This marked increase in the antigen systems level of expression, which is unexpected, could produce a boost in the kinetics and the magnitude of the binding of the indicator cell to the solid phase, which effects assay performance (e.g., sensitivity, test time etc.).

In one exemplary embodiment of the present invention, anti-A antibodies (of high affinity and concentration) can be purified using column affinity purification (i.e., immunoaffinity chromatography). In this embodiment, synthetic A antigen with an amine handle is coated on a commercially available affinity column. Source plasma from a human donor of blood group B, is then exposed to the column, where anti-A antibodies attach to the A antigen on the solid support. The anti-A antibodies are then eluted from the affinity column and can then be further purified via well-known precipitation or affinity techniques. Group A red blood cells (having A antigen) are then exposed to this purified product (i.e., IgG), and the purified anti-A IgG antibodies bind to and coat the red blood cells. Since the anti-IgG antibodies have a specificity for the IgG antibodies, they attach to the IgG antibodies, coating them partially or fully. Thus, more densely functionalized indicator cells are produced, which, in conjunction with other components, can be used to detect the presence or absence of antibodies or antigens, by using the A, B, AB and MNS antigens.

In another exemplary embodiment consistent with the present invention, as human red blood cells have a multitude of antigen systems, one may simultaneously target multiple antigen systems in order to increase the density of the IgG coating.

In another exemplary embodiment, the present invention reduces constraints on the wash cycle. To ensure that the anti-IgG antibody is not saturated by residual IgG antibodies, antigen systems that are highly expressed are chosen such that even if some quantity of the anti-IgG is saturated (and therefore, rendering it inactive), sufficient numbers of anti-IgG remain such that specific binding can be detected.

In another exemplary embodiment, plasma, protein, etc., are introduced into a solid phase system and incubated at 37° C. The solid-phase substrate is coated with human red blood cells with a known antigen profile. An antibody (in this case, IgG class) existing in test plasma with specificity for one of the antigens existing on the solid phase (the K antigen of the Kell blood group, for instance) will become specifically bound to the red blood cells on the substrate. The system is then washed with a solution to remove the unbound protein. Indicator red blood cells with a number of binding sites (probe red blood cells with anti-IgG antibody coated thereon), are introduced, and the anti-IgG antibodies (either pre-coated onto the indicator or simply added with the IgG-coated indicator) bind to the IgG on the red blood cells on the substrate.

The present invention may also be used in solution, where agglutination would be looked for, to determine if there is binding.

Thus, there has been outlined, some features that are consistent with the present invention in order that the detailed description thereof that follows may be better understood, and in order that the present contribution to the art may be better appreciated. There are, of course, additional features consistent with the present invention that will be described below and which will form the subject matter of the claims appended hereto.

In this respect, before explaining at least one embodiment consistent with the present invention in detail, it is to be understood that the invention is not limited in its application to the details of construction and to the arrangements of the components set forth in the following description or illustrated in the drawings. Methods and apparatuses consistent with the present invention are capable of other embodiments and of being practiced and carried out in various ways. Also, it is to be understood that the phraseology and terminology employed herein, as well as the abstract included below, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the methods and apparatuses consistent with the present invention.

### BRIEF DESCRIPTION OF THE DRAWINGS

FIG. 1a shows a conventional process, where an antigen system uses the D antigen.

FIG. 1b shows one embodiment consistent with the present invention, where an antigen system uses the A, B, AB, and MNS antigens.

FIGS. 2(a)-(e) shows one embodiment consistent with the present invention, where immunoaffinity chromatography is used in the purification of antibodies from blood plasma. The method uses the A, B, AB and MNS antigens.

FIGS. 3(a)-(c) shows another embodiment consistent with the present invention, where plasma and protein, etc., are introduced into a solid phase system and the substrate is coated with human red blood cells with a known antigen profile. Indicator red blood cells with anti-IgG antibodies bind to the IgG on the red blood cells on the substrate.

### DESCRIPTION OF THE INVENTION

In the present invention, antigen systems that present higher levels of expression are targeted by the inventors in order to produce more densely functionalized indicator cells which can be used to indicate the presence or absence of a particular antibody or antigen, since using the A, B, AB and MNS antigens have a remarkably greater degree of expression than the conventionally used D antigen. Such antigen systems of the present invention may include the A, B, or AB antigens or the MNSs antigens. As one of ordinary skill in the art would know, the ABO blood group system is the most important human blood group system, and includes Groups A, B, AB and O. The ABO antigens are expressed on the ends of long polylactosamine chains attached mainly to band 3 protein, the anion exchanger of the red blood cell (RBC) membrane, and a minority of epitopes are expressed on neutral glycosphingolipids.

The MNS antigen system is a human blood group system based on the glycophorin A and B genes on chromosome 4. The most important of the 46 antigens are the M, N, S, s, and U antigens.

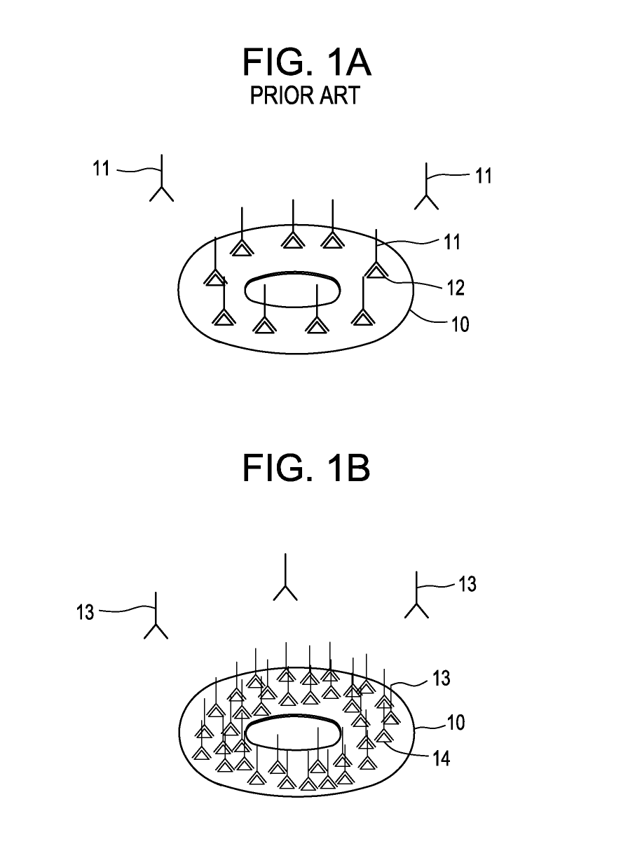

These antigen systems of the present invention, where, for example, there is a high affinity for anti-A 13, etc., antibodies (see FIG. 1b), have remarkable levels of expression that approach 1 million antigens per red blood cell 10 (potentially increasing the number of IgG sites by a factor 100). Thus, A antigen sites of about 1 million antigens 14 per red blood cell 10 is in great contrast to the conventional D antigen sites of about 10,000-30,000 antigens 12 per cell 10 (see FIG. 1a).

This remarkable increase in the antigen systems level of expression could produce a boost in the kinetics and the magnitude of the binding of the indicator cell to the solid phase, which can produce an improvement in assay performance.

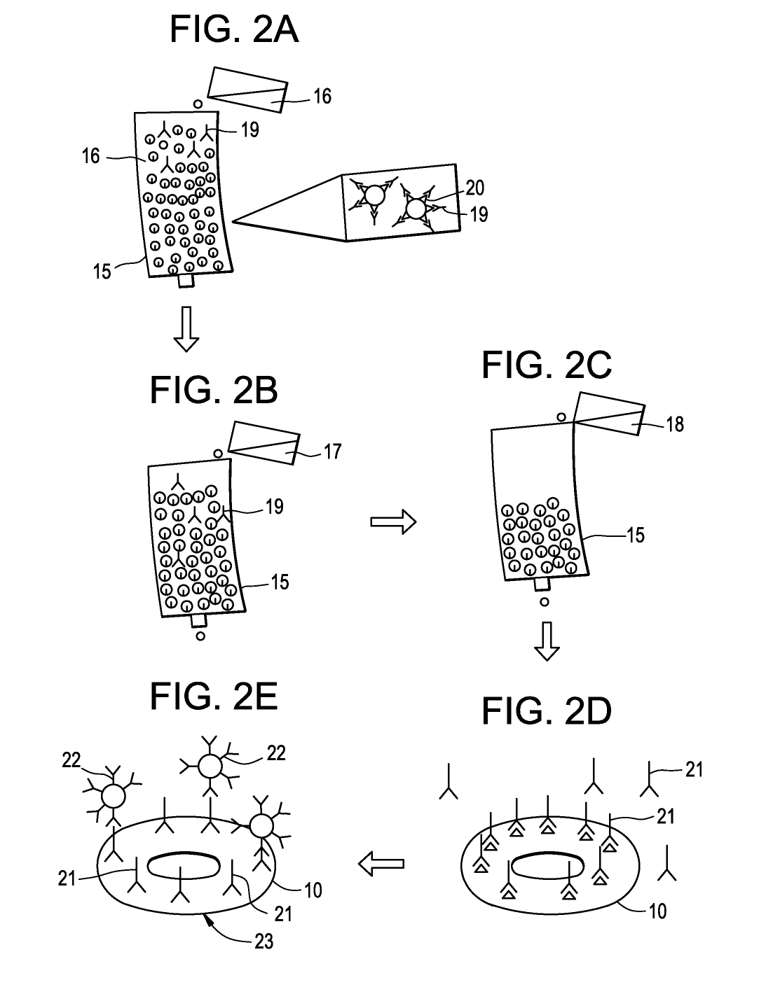

In one exemplary embodiment of the present invention, anti-A antibodies (of high affinity and concentration) can be purified as shown in FIG. 2, using column affinity purification (i.e., immunoaffinity chromatography).

In immunoaffinity chromatography, binding to the solid phase may be achieved by column chromatography whereby the solid medium is packed onto a column 15, the initial mixture 16 run through the column 15 to allow setting (FIG. 2(a)), a wash buffer 17 is run through the column 15 (FIG. 2(b)) and the elution buffer 18 subsequently applied to the column 15 (FIG. 2(c)) and collected. These steps are usually done at ambient pressure.

In the procedure, the affinity purification of antibodies 19 from human blood 16 is performed—for example, using human plasma 16 obtained from a donor of blood group B (i.e., a naturally occurring antibody) (see FIG. 2(a)). Thus, if the human plasma 16 is known to contain antibodies 19 against a specific antigen 20, then it can be purified using affinity purification. The antigen corresponding to the specificity of interest 19 can then be covalently coupled to the solid support—such as agarose—and used as an affinity ligand in purifications of antibody 19 from the human plasma 16. Affinity separation/conjugation of carbohydrates (i.e., A antigen) to column is disclosed in, for example, “Protein Purification” by Robert K. Scopes, Third Edition, Springer, NY, N.Y. 1993, and “Bioconjugate Techniques” by Greg T. Hermanson, Second Edition, Elsevier, NY, N.Y. 20008, which are herein incorporated by reference.

In this exemplary embodiment of the present invention, in step 100, synthetic A antigen 20 with an amine handle is conjugated to a commercially available affinity column 15. The column 15 is coated with the relevant A antigen 20.

In step 101 (see FIG. 2(a)), a crude product of source plasma 16 from a human donor of blood group B, is then exposed to the column 15, where anti-A antibodies 19 attach to the A antigen 20 on the solid support.

In step 102, the anti-A antibodies 19 are then purified via well-known precipitation or affinity techniques, as disclosed in “Protein Purification” by Robert K. Scopes (above), to remove the antibodies 19 of interest. Those techniques include a wash 17 (see FIG. 2(b)), and an elution of the antibodies 19 of interest using a low pH buffer 18 such as glycine pH 2.8 (see FIG. 2(c)). The eluate is collected into a neutral tris or phosphate buffer, to neutralize the low pH elution buffer and halt any degradation of the antibody's 19 activity. This procedure removes the undesirable antibodies 19 from the human plasma 16 and purifies the target antibody 19.

The anti-A antibodies 19 can be further separated to IgG or IgM antibodies 21 (for instance, a protein G column will selectively bind IgG but not IgM), using the same techniques described above.

Thus, in step 103, group A red blood cells 10 (having A antigen 20) are then exposed to this purified product (i.e., IgG antibody 21) (see FIG. 2(d)), and the purified product of IgG antibodies 21 bind with and coat the red blood cells 10.

In step 104, the coated cells 10 are then washed according to known methods, as described in Sinor et al.

In step 105, the cells 10 are then exposed to anti-IgG antibodies 22. Since the anti-IgG antibodies 22 have a specificity for the IgG antibodies 21, they attach to the IgG antibodies 21, coating the indicator cells 23 (see FIG. 2(e)). This step may be conducted prior to use (i.e., pre-sensitizing the IgG coated red cells with anti-IgG) or during use (simply mixing the IgG coated indicators with anti-IgG).

Thus, the present invention discloses a novel and unexpected procedure which should produce more densely functionalized indicator cells 23 which, along with other components, can be used to indicate the presence or absence of antibodies or antigens, by using the A, B, AB and MNS antigens which have a greater degree of expression than the D antigen.

In another embodiment consistent with the present invention, as human red blood cells have a multitude of antigen systems, one may simultaneously target multiple antigen systems in order to increase the density of the IgG antibody coating, for example.

For example, one may choose red blood cells that express Rh, Kell, and Duffy antigens and expose these red blood cells to a blend of antibodies with specificities towards these groups. Hence, by targeting multiple groups simultaneously, one can obtain higher coating densities.

The technique of the present invention may also reduce constraints on a wash cycle as well. Many conventional assays that rely upon class-specific immunoglobulin (i.e., anti-IgG antibodies) require that unbound antibodies 19 be removed from the system (see FIG. 2(b), for example)). If this is not the case, the anti-IgG antibody 22 (see FIG. 2(e)) will become saturated with “generic” IgG antibody 21 and specific binding will not be detected. This effect is prevented by washing the system with a solution that does not contain free IgG antibody 21.

However, since this wash process can be somewhat demanding if ultra-low levels of residual IgG antibody 21 are required, it is important to ensure that the anti-IgG antibody 22 is not saturated by residual IgG antibodies 21. This can be accomplished by choosing antigen systems that are highly expressed such that even if some quantity of the anti-IgG antibody 22 is saturated (and therefore, rendering it inactive), sufficient numbers of anti-IgG antibody 22 remain such that specific binding can be detected.

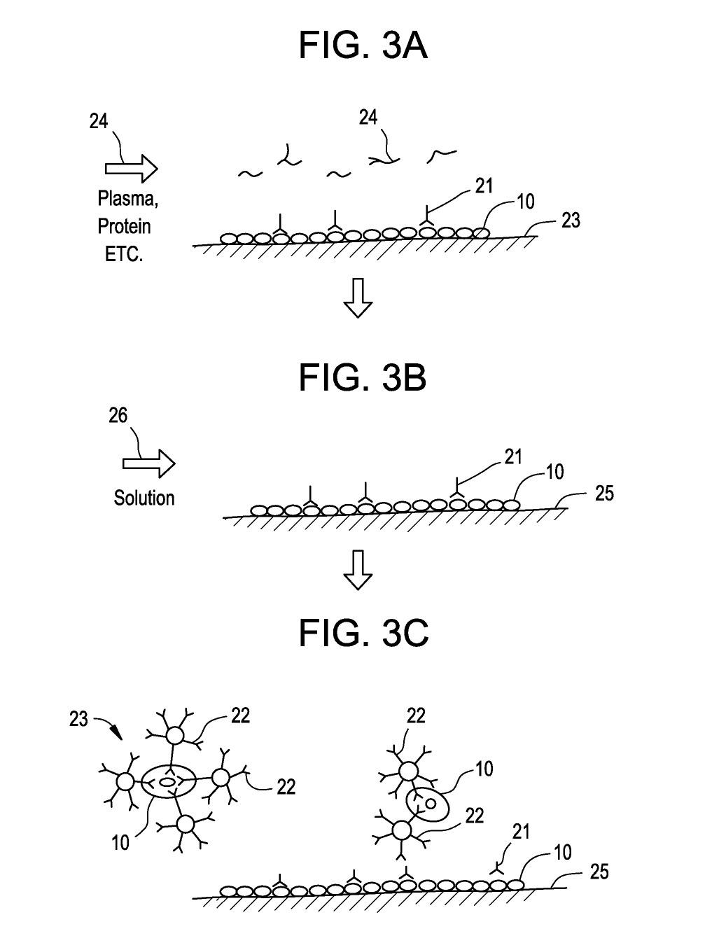

In one exemplary embodiment consistent with the present invention, FIG. 3(a), in step 200, shows where plasma, protein, etc., 24 are introduced into a solid phase system and incubated at 37° C. The substrate 25 is coated with human red blood cells 10 with a known antigen profile.

In step 201, the specific antibody 21 desired (in this case, IgG antibody 21), becomes specifically bound to the red blood cells 10 (i.e., the K antigen, of the Kell system) on the substrate 25.

In step 202 (see FIG. 3(b)), the system is washed with a solution 26 (i.e., saline) to remove the unbound protein. Since plasma 24 contains a large number of different kinds of antibodies, it is important to have a good washing step to remove any residual IgG antibody 21 left free in the system.

In step 203 (see FIG. 3(c)), indicator red blood cells 23 with a number of binding sites (probe red blood cells 10 with anti-IgG antibody 22 coated thereon, produced from the methods described above), are introduced into the system, and the anti-IgG antibodies 22 bind to the IgG antibodies 21 on the red blood cells 10 on the substrate 25. As noted above, if there is too much IgG antibody 21 from the plasma 24 in the system, the probe red blood cells 23 will become coated and prevent binding at the substrate 25; thus, it is important that there is careful washing of the system, as well as using probe indicator cells 23 with many binding sites.

The present invention may also be used in solution, where agglutination would be looked for on the solid phase, to determine if there is binding.

It should be emphasized that the above-described embodiments of the invention are merely possible examples of implementations set forth for a clear understanding of the principles of the invention. Variations and modifications may be made to the above-described embodiments of the invention without departing from the spirit and principles of the invention. All such modifications and variations are intended to be included herein within the scope of the invention and protected by the following claims.

What is claimed is:

1. A method of purifying antibodies in a solid phase antibody screening assay, to produce dense functionalized indicator cells which can be used as a component of an assay that will indicate a presence or an absence of antibodies or antigens, comprising:
coating a predetermined antigen to an affinity column, said predetermined antigen being one of A, B, AB and MNS antigens;
introducing human plasma into said affinity column, such that antibodies disposed in said human plasma attach to said predetermined antigen;
purifying said antibodies using immunoaffinity chromatography, such that said purified antibodies remain and other undesired antibodies are removed;
exposing said purified antibodies to red blood cells, such that said purified antibodies bind with and coat said red blood cells; and
forming indicator cells by exposing said coated red blood cells to additional antibodies that have a specificity for said purified antibodies, such that said additional antibodies attach to said purified antibodies and coat said red blood cells.

2. The method of claim 1, wherein said predetermined antigen is A antigen, and said antibodies are anti-A antibodies.

3. The method of claim 2, further comprising:
separating said anti-A antibodies into IgG antibodies or IgM antibodies using immunoaffinity chromatography purification.

4. The method of claim 3, wherein said purified product is IgG antibody, and said additional antibodies are anti-IgG antibodies.

5. The method of claim 1, further comprising:
washing said coated cells before exposing them to said additional antibodies.

6. The method of claim 1, wherein said purifying step comprises:
washing said human plasma; and
eluting said predetermined antibodies using a buffer.

7. The method of claim 1, wherein more than one antigen is used in said affinity column to increase a density of said antibody coating on said red blood cells.

8. The method of claim 7, wherein said more than one antigen includes Rh, Kell, Kidd, and Duffy antigens.

9. The method of claim 6, wherein said washing includes a solution that does not contain free predetermined antibodies.

10. The method of claim 1, further comprising:
introducing at least plasma and protein into a solid phase system having a substrate coated with red blood cells with a predetermined antigen profile, such that said predetermined antibody binds to said red blood cells on said substrate;
washing said system with a buffer solution to remove unbound protein and residual predetermined antibody; and
introducing said indicator cells with said additional antibodies coated thereon, such that said additional antibodies bind to said predetermined antibodies bound on said red blood cells on said substrate.

11. The method of claim 10, further comprising:
Incubating said at least plasma and protein introduced into said solid phase system, at 37° C.

12. The method of claim 10, wherein said additional antibodies coated on said indicator cells are anti-IgG antibodies, and said predetermined antibodies are IgG antibodies, and said anti-IgG antibodies bind to said IgG antibodies.

13. The method of claim 10, wherein agglutination is looked for to determine when there is binding.

14. The method of claim 1, wherein a level of expression of said predetermined antigen is 10,000-30,000 antigens per red blood cell.
